(19) United States
(12) Patent Application Publication
(10) Pub. No.: US 2010/0055669 A1
(43) Pub. Date: Mar. 4, 2010

(54) Generation of Recombinant Genes in Bacteriophages

(75) Inventors: Alejandro Luque, Paris (FR); Heike Strobel, Vienna (AT); Jann Thorsten Martinsohn, Vanves (FR); Marie-Agnès Petit, Paris (FR); Miroslav Radman, Gentilly (FR)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: MIXIS FRANCE, S.A., Paris (FR)

(21) Appl. No.: 11/570,608

(22) PCT Filed: Jul. 6, 2005
(86) PCT No.: PCT/EP2005/007291

§ 371 Date: Jul. 31, 2008

(30) Foreign Application Priority Data
Jul. 6, 2004 (EP) 04360062.6

(51) Int. Cl. C12Q 1/70; C12P 21/02; C12N 7/01; C12N 15/74; C07K 14/00; C07H 21/04

(52) U.S. Cl. 435/5; 435/69.7; 435/235.1; 435/320.1; 530/350; 536/23.1

(57) ABSTRACT

In vivo methods for generating and detecting recombinant DNA sequences in bacteriophages or plasmids containing bacteriophage sequences, methods for generating hybrid genes and hybrid proteins encoded by these hybrid genes by the use of bacteriophages and plasmids containing bacteriophage sequences, bacteriophages and plasmids that can be used in these methods, and kits comprising appropriate bacterial host cells and bacteriophages or plasmids are described.

The present invention relates to in vivo methods for generating and detecting recombinant DNA sequences in bacteriophages or plasmids containing bacteriophage sequences, methods for generating hybrid genes and hybrid proteins encoded by these hybrid genes by the use of bacteriophages and plasmids containing bacteriophage sequences, bacteriophages and plasmids that can be used in these methods, and kits comprising appropriate bacterial host cells and bacteriophages or plasmids. DNA sequences for which these methods are relevant include protein-encoding and non-coding sequences.

Traditional mutagenesis approaches for evolving new properties in enzymes, such as site-directed mutagenesis, random mutagenesis and error prone PCR, have a number of limitations. These approaches are only applicable to genes or sequences that have been cloned and functionally characterized and that have a discrete function. Also, the traditional mutagenesis approaches can only explore a very limited number of the total number of permutations, even for a single gene. However, under certain circumstances it might be necessary to modify not only one gene, but additional genes, in order to express a protein, with new properties. Such additional genes can be for example genes that cooperatively confer a single phenotype or genes that have a role in one or more cellular mechanisms such as transcription, translation, post-translational modifications, secretion or proteolytic degradation of a gene product. Attempting to individually optimize all of the genes having such function by traditional mutagenesis approaches would be a virtually impossible task.

Furthermore, numerous conventional mutagenesis approaches are based on the use of genetic engineering methods, such as restriction and ligation. However, the restriction-ligation approach has several practical limitations, namely that DNA molecules can be precisely combined only if convenient restriction sites are available and that, because useful restriction sites often repeat in a long stretch of DNA, the size of DNA fragments that can be manipulated are limited, usually to less than about 20 kilobases.

Most of the problems associated with conventional mutagenesis approaches can be overcome by recombination approaches which entail randomly recombining different sequences of functional genes, enabling the molecular mixing of naturally similar or randomly mutated genes. Due to its experimental simplicity and the freedom from DNA-sequence imposed limitations recombination provides an alternative method for engineering DNA. Also, by using recombination approaches the probability of obtaining mutants with improved phenotype is significantly higher than by applying conventional mutagenesis methods including genetic engineering techniques.

Recombination is tightly coupled with DNA replication and repair. This tight interrelationship between recombination and DNA replication was first evident in the bacteriophage T4 and the related T-even phages. Because DNA of T4 and its host Escherichia coli differ in base composition and modifications and because the host DNA is rapidly degraded after phage infection, molecular aspects of T4 replication and recombination could be readily investigated by biochemical, biophysical, and genetic methods. Early characterization of mutations in most essential genes and the almost complete dependence of replication and recombination on phage-encoded proteins allowed analysis of recombination and replication proteins, as well as “reality checks” of results obtained with genetic and biochemical methods.

Despite the detailed characterization of recombination in bacteriophages, in contrast to unicellular organisms such as bacteria or yeast, where numerous different systems for effecting recombination exist, only a few bacteriophage-based systems for effecting recombination are known, which can be used for the generation of new mosaic or hybrid genes. However, most of these phage-based systems have several drawbacks. In particular, most of the bacteriophage-based systems do not allow an easy and efficient detection of newly recombined DNA sequences.

Therefore, there is still in the art a demand for efficient bacteriophage test systems, which in particular allow a rapid and simple detection of recombinants and/or a selection of recombinants under selective pressure.

The technical problem underlying the present invention is therefore to provide improved methods and means for a simple and efficient generation of recombinant mosaic genes in bacteriophage systems, in particular for screening and detecting such recombinant sequences.

The present invention solves this underlying technical problem by providing a process for generating and detecting recombinant DNA sequences in a system comprising a bacteriophage and a bacterial host cell, wherein bacteriophage contains a promoter flanked by a first and a second DNA sequences to be recombined and at least a first marker gene, located downstream of the first DNA sequence, wherein recombination between the two DNA sequences leads to an inversion of the promoter in a flip-flop manner and wherein depending on the orientation of the promoter one or the other of the DNA sequences and at least first marker gene can be transcribed or not, comprising the steps of:
a) incubation of a first host cell containing the bacteriophage under selective conditions, that only allow the propagation of the cell and/or of the bacteriophage if the promoter is oriented such that the gene product of the first marker gene is expressed, and
b) isolation of the bacteriophage progeny derived from the first host cells grown and/or propagated under selective conditions and containing a first and a second recombined DNA sequences.

The present invention provides a system based on bacteriophages to screen for recombination events between at least two divergent DNA sequences or recombination substrates in vivo. The inventive system allows the generation of new advantageous DNA sequences with improved properties in a fast and efficient way by a process involving an in vivo exchange of DNA from two recombination substrates, i.e. two divergent DNA sequences to be recombined which are located in inverted orientation on a bacteriophage. On that bacteriophage these two recombination substrates flank a promoter in a given configuration. This promoter can change its orientation in a flip-flop manner. Depending on the orientation of the promoter, one or the other of the two recombination substrates is transcribed, and marker genes further downstream of the recombination substrates are similarly under this transcriptional control. The expression of these downstream marker genes can be detected and selected for under appropriate conditions, thereby allowing a specific promoter orientation to be selected. Since crossover recombination involving the two recombination substrates leads to promoter inversion, recombinants can be identified under conditions that select for the expression of specific downstream marker genes.

In the inventive recombination process the host cell, comprising the bacteriophage is incubated under such conditions, which select for the presence of the gene product of the first marker gene. The selective conditions employed include such conditions which, prevent the growth and/or propagation of the host cell and thus also the propagation of the bacteriophage, if the gene product of the first marker gene is expressed. This means that propagation of the host cells and propagation of the bacteriophage only occur if the first marker gene is transcribed from the promoter present on the inventive vector, meaning that the promoter must have inverted its orientation due to recombination between the two DNA sequences to be recombined such that it can direct the transcription of the first marker gene. Therefore, recombination events can easily be followed up by incubation of the host cells under selective conditions which select for the inversion of the promoter and thus for the generation of recombined DNA sequences.

However, the inventive process has the advantage that it is iterative, i.e. it allows further rounds of recombination. These further rounds of recombination are based on the inversion of the promoter due to crossover recombination involving the two recombination substrates. The inversion of the promoter in the second round of recombination has the result that the first marker gene cannot be transcribed anymore. However, the promoter inversion renders possible that other marker genes located on the other side of the promoter relative to the first marker gene now can be transcribed. Therefore, the bacteriophage progeny containing the products of the first round of recombination, i.e. the first and second recombined DNA sequences, can again be introduced in appropriate host cells in order to effect a second round of recombination. In one embodiment of the inventive process the host cells containing the phage progeny obtained in the first round of recombination are incubated under such conditions, which select for the absence of the gene product of the first marker gene. In another embodiment the host cells containing the phage progeny obtained in the first round of recombination are incubated under such conditions, which select for the presence of the gene product of a second marker gene. In this way many rounds of recombination can be conducted by simply changing the selective conditions and selecting for the alternating inversion of the promoter.

Thus, the inventive process provides an easy and quick selection system to identify recombinant DNA sequences, which is based on the alternate expression of marker genes, depending on the orientation of the promoter.

According to the invention two selection strategies were developed in order to create and detect mosaic genes with a high efficiency in vivo. These selection strategies are based on the different life cycles of lytic and temperate phages and allow for the detection of recombinants during the lytic phase or as bacterial lysogens. In case of detection during the lytic phase of phage development the selection is based, for example, on the expression or absence of expression of one or more genes of the phage itself, such as the lambda gam gene. In one orientation of the intervening sequence, transcription from the promoter activates for example the gam gene, which allows plaque formation on an E. coli recA lawn and prevents plaque formation on an E. coli P2 lysogen lawn. When the promoter is present in the opposite orientation, the absence of gam transcription allows lytic growth on the P2 lysogen and prevents growth on the recA host.

However, recombinants can also be recovered as bacterial lysogens, i.e. cells that harbor the bacteriophage genome in their chromosome in the form of a prophage, rather than as plaques. Instead of activating transcription of the gam gene, in one orientation the promoter can activate a gene expressing an antibiotic resistance marker and in the other orientation it activates another gene expressing a different antibiotic resistance marker.

By using the two different inventive selection strategies it was surprisingly found, that bacteriophages, in particular bacteriophage lambda, effectively recombine diverged sequences, with frequencies ranging from 10⁻³ to 10⁻⁶, depending on the extent of divergence. This is especially true for recombination using the lambda red and gam genes as marker genes, where recombination frequencies of 10⁻³ were obtained. Even higher frequencies are obtained if mismatch repair deficient host cells, such as E. coli ΔmutS mutants are used. The results obtained by the inventive process are surprising, since to date it was only known that bacteriophages can recombine very similar, nearly identical sequences, as described by Kleckner and Ross (J. Mol. Biol., 144 (1980), 215-221). However, nothing was known about the ability of bacteriophages to recombine diverged sequences, in particular greatly diverging sequences.

Therefore, the inventive process for generating and detecting recombined DNA sequences in bacteriophages has the advantage that greatly diverging DNA sequences can be recombined. Unexpectedly, it was found that sequences with a high degree of overall divergence and which only share very short stretches of homology or identity can be recombined. An analysis of recombined sequences revealed that the stretches of identity in which recombination occurred can comprise only a few nucleotides, for example less than 10 nucleotides. The diversification of the recombination substrates achieved by the use of the inventive process is remarkably very efficient. No obvious recombination hotspots could be identified. Only in three cases out of 42 “flip” recombinants identical recombination products were identified, all of them were obtained by recombining the same two diverging sequences, namely Oxa7 and Oxa5.

Advantageously, the inventive process can be conducted either in wild-type or mismatch repair-defective bacterial host cells. The processes by which damaged DNA is repaired and the mechanisms of genetic recombination are intimately related, and it is known that the mismatch repair machinery has inhibitory effects on the recombination frequency between divergent sequences, i.e. homeologous recombination. Mutations of the mismatch repair system therefore greatly enhance the overall frequency of recombination events in bacterial cells. According to the invention it was found that, if mismatch repair-defective bacterial host cells are used for the inventive process, the frequency of recombination can substantially be increased. For example the frequency of recombination was about ten times higher in a ΔmutS mutant of E. coli than in the corresponding wild-type cell. Furthermore, it was found that if the inventive process is carried out in a mismatch repair-defective background such as in a mutS background, recombination is accompanied by the introduction of point mutations contributing in addition to the generation of new mosaic genes.

Together with the diversification of the substrate sequences used, observed on the sequence level, the results obtained by the use of the inventive process show that the bacteriophage tools provided by the present invention can be exploited to create large libraries of diversified genes in directed evolution experiments. With the inventive process large libraries of recombined, mutated DNA sequences can be easily generated, and variants that have acquired a desired function can then be identified by using an appropriate selection or screening system.

The inventive use of bacteriophages for effecting recombination processes has furthermore the obvious advantage of the ease of manipulation of DNA sequences and the possibility of studying specific recombination events induced synchronously in a large population of bacteriophages. Thus, by the use of bacteriophages it is possible to conduct many rounds of recombination within a short time and to create a plurality of new recombinant DNA sequences.

A preferred embodiment of the inventive process for generating and detecting recombinant DNA sequences in bacteriophages relates to a second round of recombination and comprises the steps of:
a) introduction of the bacteriophage progeny obtained in 1b) into a second bacterial host cell,
b) incubation of the second host cell containing the bacteriophage progeny under selective conditions, that only allow the propagation of the cell and/or of the bacteriophage if the promoter is oriented such that the gene product of the first marker gene is not expressed, and
c) isolation of the bacteriophage progeny derived from the second host cells grown and/or propagated under selective conditions and containing a third and a fourth recombined DNA sequences.

In another preferred embodiment of the inventive process further recombined DNA sequences are generated by subjecting the bacteriophage progeny obtained in the second recombination round at least once to another cycle of steps to effect a first round of recombination or steps to effect first and second rounds of recombination.

According to the invention the first and/or second bacterial host cells containing bacteriophages are generated by the introduction of bacteriophage that comprises the two recombination substrates flanking the promoter and the first marker gene, into a suitable bacterial cell, thereby allowing the bacteriophage to follow either a lytic or a lysogenic life cycle. In the context of the invention a “bacteriophage” is a virus with both living and nonliving characteristics, that only infects bacteria. In particular the phage consists of DNA. There are two primary types of phages, namely lytic phages and temperate phages. Lytic phages that replicate through the lytic life cycle terminate their infection and breach the envelope of the host cell, i.e. lyse the host bacterium, in order to release their progeny into the extracellular environment. A temperate phage is one that is capable of displaying a lysogenic infection. A lysogenic infection is characterized in that the host bacterium containing the phage does not produce nor release phage progeny into the extracellular environment. Instead, the genetic material of the phage inserts or integrates into the DNA of the host bacterium. The genetic material of the phage is propagated together with the DNA of the host bacterium. A temperate phage typically displays a lytic cycle as its vegetative, i.e. non-lysogenic, phase. The host cell used according to the invention for introducing the bacteriophage can be either a cell that does not contain a prophage or a cell that already contains in its genome a prophage, i.e. a bacterial lysogen. In the latter case the prophage and the bacteriophage introduced share preferably some homologous sequences such that the bacteriophage introduced can be integrated by recombination into the genome of the host cell.

In a particularly preferred embodiment of the inventive process the bacteriophage used is bacteriophage lambda. The lambda phage is a temperate phage which either can display a lytic or lysogenic infection. The lambda phage has its own recombination system (red). Characteristics of Red-mediated recombination in lambda crosses are a break-and-join mechanism, non-reciprocal DNA exchange and a heteroduplex length of about 10% of the total genome. However, lambda can recombine by the host recombination system if its own recombination genes are mutant. In crosses with red⁻ gam⁻ phage, recombination uses the recA and recBC genes of E. coli. Characteristics are a break-and-join mechanism, probably a reciprocal exchange of DNA and usually hotspots for recombination.

In another embodiment of the invention the first bacterial host cells containing bacteriophages are generated by introducing a plasmid containing bacteriophage sequences, the two DNA sequences to be recombined which flank the promoter and the at least first marker gene, into a bacterial lysogen, i.e. a cell containing a prophage in its genome. The prophage preferably contains sequences that are homologous to the bacteriophage sequences contained in the plasmid in order to enable the integration of at least that part of the plasmid that comprises the promoter and the two flanking recombination substrates plus the first marker gene into the genome of the host cell. In another preferred embodiment of the invention a linear sequence from such a plasmid is introduced into a bacterial lysogen in order to generate the first bacterial host cell.

In a particularly preferred embodiment of the inventive process the plasmid used is plasmid pMIX-LAM, which is a derivative of plasmid pACYC184 that contains the pL+N promotor region and the flanking sequences cI+rexa and cIII+IS10 of bacteriophage lambda. pMIX-Lam contains furthermore a Cm^{R} gene. The vector also contains the multicloning sites MCS1 and MCS2, which flank the promoter-containing pL+N fragment of lambda for inserting foreign DNA sequences. Plasmid DNA containing two DNA sequences to be recombined is cut with appropriate restriction enzymes in the lambda flanking regions cI and cIII to yield a fragment that contains the recombination substrates and that can be targeted to the lambda genome in a recipient host lysogen.

In still another particularly preferred embodiment of the inventive process the vector used is plasmid pAC-OX-OY, which is derived from a low copy number plasmid and which contains the colE1 replication origin. Plasmid pAC-OX-OY furthermore contains the two resistance markers Spec^{R} and Cm^{R}, which flank the two recombination substrates and the targeting sequences LG and LD located at the ends of the recombination substrates. The targeting sequences promote integration into a lambda prophage genome. Linear DNA fragments containing the recombination substrates are obtained by enzymatic restriction and purification or by PCR amplification of the cassette.

In the context of the present invention a “promoter” is a DNA region located upstream of a DNA sequence such as a protein-coding sequence and to which a RNA-polymerase can bind. If the promoter is correctly oriented, then transcription of the downstream located DNA sequence can be initiated. According to the invention the promoter is flanked by two non-identical DNA sequences to be recombined in an inverted configuration. Recombination between these two DNA sequences leads to an inversion of the promoter. Another recombination between the two flanking DNA sequences leads again to a promoter inversion whereby the promoter flips back into its original orientation. Thus, the promoter used in the present invention is subjected to a flip-flop mechanism by which the promoter orientation is inverted in each recombination round. In a preferred embodiment of the inventive process the promotor is the pL promoter of lambda. In another preferred embodiment of the inventive process the promotor is the artificial promoter Pro.

According to the invention the bacteriophage or plasmid used to generate the first bacterial host cell contains at least one marker gene, i.e. the first marker gene. In the context of the present invention the term “marker gene” refers to an unique protein-coding DNA sequence that is located only on the bacteriophage or plasmid used, but nowhere else in the genome of the host cell, and that is positioned on the bacteriophage or plasmid downstream of one of the two recombination substrates or one of the two already recombined DNA sequences and downstream of the promoter used. The presence of one or more marker genes on the same DNA molecule as the recombination substrates or already recombined DNA sequences allows recombination events leading to recombined DNA sequences to be recognized and selected for, in particular by genetic methods.

According to the invention the first marker gene is located downstream of the first DNA sequence to be recombined and also downstream of the promoter. This arrangement allows for the selection of crossovers involving two recombination substrates, i.e. two DNA sequences to be recombined, since recombination between the first and the second DNA sequences leads to an inversion of the promoter, whereby depending on the orientation of the promoter the first marker gene can be transcribed or not. The presence or absence of the gene product of the first marker gene therefore can be used to select for recombination events. This arrangement also allows further rounds of recombination to be carried out in an iterative fashion.

In a preferred embodiment of the invention the first marker gene is selected from the group consisting of a lambda gene, a nutritional marker gene, an antibiotic resistance marker gene and a sequence encoding a subunit of an enzyme.

A “nutritional marker” is a marker gene that encodes a gene product that can compensate an auxotrophy of an organism or cell and thus can confer prototrophy on that auxotrophic organism or cell. In the context of the present invention the term “auxotrophy” means that an organism or cell must be grown in a medium containing an essential nutrient which cannot be synthesized by the auxotrophic organism itself. The gene product of the nutritional marker gene promotes the synthesis of this essential nutrient missing in the auxotrophic cell. Therefore, upon expression of the nutritional marker gene it is not necessary to add this essential nutrient to the medium in which the organism or cell is grown, since the organism or cell has acquired prototrophy.

An “antibiotic resistance marker” is a marker gene wherein the gene product confers upon expression to a cell, in which the expression of the antibiotic marker gene takes place, the ability to grow in the presence of a given antibiotic at a given concentration, whereas a cell without the antibiotic resistance marker cannot.

A “sequence encoding a subunit of an enzyme” can be used as a marker gene, if a cell cannot synthesize all subunits of an enzyme that are required for the assembly of the complete enzyme structure and thus for obtaining the full activity of the enzyme, and if the presence or absence of the enzymatic activity can be monitored by genetic means. If, for example, the activity of an enzyme is needed for an essential biochemical pathway of the cell, which enables the growth and/or propagation of the cell in a particular environment, and the cell cannot synthesize all components of the complete enzyme structure, then the cell cannot survive in that environment. The “sequence encoding a subunit of an enzyme” used as marker gene therefore allows upon expression the assembly of the complete enzyme and the survival of the cell.

In a particular preferred embodiment of the invention the first marker gene is the gam gene of lambda. The gam gene belongs together with redX (or exo) and redβ to that three genes of lambda that affect recombination. Without Gam, lambda cannot initiate rolling circle replication because RecBCD degrades the displaced linear end of DNA. In the inventive process the transcription of the gam gene from the promoter, in particular pL, allows the formation of plaques on a lawn of Escherichia coli recA host cells and prevents plaque formation on a lawn of E. coli P2 lysogenic host cells. In contrast, the absence of transcription of the gam gene due to an inverted orientation of the promoter, in particular pL, allows the plaque formation on a lawn of E. coli P2 lysogenic host cells and prevents the plaque formation on a lawn of E. coli recA host cells.

In a particular preferred embodiment of the invention the first marker gene is Cm^{R}, the gene product of which confers a cell resistance to chloramphenicol. Therefore, in the inventive process transcription of the Cm^{R} gene from the promoter, in particular Pro, in one orientation allows the growth of the bacterial host cells on a medium containing chloramphenicol, whereas the absence of transcription of the Cm^{R} gene due to the inverted orientation of the promoter, in particular Pro, prevents the growth of the bacterial host cells on a medium containing chloramphenicol.

In another preferred embodiment of the invention more than one marker can be located on the bacteriophage or plasmid used, whereby additional markers are introduced to increase the stringency of selection. According to the invention the bacteriophage or plasmid used can contain at least a second marker gene that is located downstream of the second DNA sequence to be recombined and also downstream of the promoter. Therefore the first and the second marker genes flank in an inverted configuration the promoter used, whereby only one of the two marker genes can be transcribed from the promoter depending on its orientation.

Preferably the second marker gene is selected from the group consisting of a nutritional marker gene, an antibiotic resistance marker gene and a sequence encoding a subunit of an enzyme.

In a particular preferred embodiment of the invention the second marker gene is Spec^{R} which is preferably combined with the Cm^{R} gene as first marker gene. The transcription of the Spec^{R} gene from the promoter, in particular Pro, allows the growth of the bacterial host cells on a medium containing spectinomycin, whereas the absence of transcription of the Spec^{R} gene due to the orientation of the promoter, in particular Pro, prevents the growth of the bacterial host cells on a medium containing spectinomycin.

According to the invention a bacterial cell is used as host cell for introducing the bacteriophage or plasmid containing the two DNA sequences to be recombined. The terms “bacterial cell” and “bacterial host cell” include any cell, in which the genome is freely present within the cytoplasm as a circular structure, i.e. a cell, in which the genome is not surrounded by a nuclear membrane. The host cell can already contain a prophage.

In a preferred embodiment of the invention the bacterial host cell is a cell of a gram-negative bacterium, in particular E. coli, a gram-positive bacterium or a cyanobacterium.

According to the invention it may be preferred to use bacterial host cells for the inventive process which have a functional repair system. The mismatch repair (MMR) system is one of the largest contributors to avoidance of mutations due to DNA polymerase errors in replication. However, mismatch repair also promotes genetic stability by ensuring the fidelity of genetic recombination. Whereas in bacteria and also in yeast and mammalian cells, recombination between homeologous DNA substrates containing a few mismatches (<1%) occurs much less efficiently than between identical sequences, the frequency of recombination (gene conversion and/or crossovers) is dramatically elevated in MMR-defective lines. This means, that the high fidelity of recombination is not only caused by the intrinsic properties of recombination enzymes, but also by the editing of recombination by the mismatch repair system. Thus the mismatch repair machinery has an inhibitory effect on recombination between diverged sequence. In E. coli two proteins of the methyl-directed MMR system, namely MutS and MutL, are required for this strong antirecombination activity, whereas the effect of the other MMR system proteins, MutH and UvrD, is less pronounced. In addition to their roles in MMR and homeologous recombination, MMR proteins also play an important role in removing non-homologous DNA during gene conversion.

In another preferred embodiment of the invention, bacterial cells that are deficient in the mismatch repair system are used. In the context of the present invention the term “deficient in the mismatch repair system” means that the MMR system of a bacterial cell is transiently or permanently impaired. MMR deficiency of a bacterial cell can be achieved by any strategy that transiently or permanently impairs the MMR system including but not limited to a mutation and/or a deletion of one or more genes involved in MMR, treatment with an agent like UV light, which results in a global impairment of MMR, treatment with an agent like 2-aminopurine or a heteroduplex containing an excessive amount of mismatches to transiently saturate and inactivate the MMR system, and inducible expression or repression of one or more genes involved in MMR, for example via regulatable promoters, which would allow for transient inactivation.

In a preferred embodiment of the invention the mismatch repair deficiency of the bacterial host cell is due to a mutation of at least one of the genes involved in MMR. In a preferred embodiment the bacterial cells have a mutated mutS gene, a mutated mutL gene, a mutated mutH gene and/or a mutated UvrD gene.

In the context of the present invention the terms “DNA sequences to be recombined” and “recombination substrate” mean any two DNA sequences that can be recombined as a result of recombination, processes. Recombination substrates can include already recombined DNA sequences. Recombination between recombination substrates can be due to homologous or non-homologous recombination. Homologous recombination events of several types are characterized by the base pairing of a damaged DNA strand with a homologous partner, where the extent of interaction can involve hundreds of nearly perfectly matched base pairs. The term “homology” denotes the degree of identity existing between the sequence of two nucleic acid molecules. In contrast, illegitimate or non-homologous recombination is characterized by the joining of ends of DNA that share no or only a few complementary base pairs.

The first and second DNA sequences to be recombined are diverging sequences, i.e. sequences which are not identical but show a certain degree of homology. This means that the DNA sequences to be recombined diverge by at least one nucleotide or at least two nucleotides. In a preferred embodiment of the invention the overall compositions of the first and the second DNA sequences to be recombined diverge by more than 0.1%, by more than 5%, by more than 10%, by more than 20%, by more than 30%, by more than 40% or by more than 50%. This means that the first and second DNA sequences to be recombined can also diverge by 55%, 60%, 65% or even more. Preferably the DNA sequences to be recombined are sequences that share at least one or more homologous regions, which can be very short. The homologous regions can have a length of about 5-50 nucleotides.

Recombination substrates or DNA sequences to be recombined can have a natural or synthetic origin. Therefore, in a preferred embodiment of the invention the first and the second DNA sequences to be recombined are naturally, occurring sequences and/or artificial sequences. Naturally occurring DNA sequences to be recombined can be derived, from any natural source including viruses, bacteria, fungi, animals, plants and humans. Artificial or synthetic DNA sequences to be recombined can be generated by any known method.

In a preferred embodiment of the invention DNA sequences to be recombined are protein-encoding sequences, for example sequences encoding enzymes, which can be utilized for the industrial production of natural and non-natural compounds. Enzymes or those compounds produced by the help of enzymes can be used for the production of drugs, cosmetics, foodstuffs, etc. Protein-encoding sequences can also be sequences, which encode proteins, that have therapeutic applications in the fields of human and animal health. Important classes of medically important proteins include cytokines and growth factors. The recombination of protein coding sequences allows for the generation of new mutated sequences which code for proteins with altered, preferably improved functions and/or newly acquired functions. In this way it is possible, for example, to achieve improvements in the thermostability of a protein, to change the substrate specificity of a protein, to improve its activity, to evolve new catalytic sites and/or to fuse domains from two different enzymes. Protein coding DNA sequences to be recombined can include sequences from different species which code for the same or similar proteins that have in their natural context similar or identical functions. Protein coding DNA sequences to be recombined can include sequences from the same protein or enzyme family. Protein coding sequences to be recombined can also be sequences which code for proteins with different functions—for example, sequences that code for enzymes which catalyse different steps of a given metabolic pathway. In a preferred embodiment of the invention the first and the second DNA sequences to be recombined are selected from the group of gene sequences of the Oxa superfamily of beta-lactamases.

In another preferred embodiment of the invention DNA sequences to be recombined are non-coding sequences such as sequences, which, for example, are involved within their natural cellular context in the regulation of the expression of a protein-coding sequence. Examples for non-coding sequences include but are not limited to promoter sequences, sequences containing ribosome binding sites, intron sequences, polyadenylation sequences etc. By recombining such non-coding sequences it is possible to evolve mutated sequences, which in a cellular environment result in an altered regulation of a cellular process—for example, an altered expression of a gene. Non-coding DNA sequences to be recombined can include sequences from different species which, for example, have in their natural context similar or identical regulatory functions.

According to the invention a recombination substrate or DNA sequence to be recombined can of course comprise more than one protein coding sequence and/or more than one non-coding sequence. For example a recombination substrate can comprise one protein coding sequence plus one non-coding sequence or a combination of different protein coding sequences and different non-coding sequences. In another embodiment of the invention DNA sequences to be recombined therefore can consist of one or more stretches of coding sequences with intervening and/or flanking non-coding sequences. That means the DNA sequence to be recombined can be for example a gene sequence with regulatory sequences at its 5′-terminus and/or an untranslated 3′-region or an mammalian gene sequence with an exon/intron structure. In still another embodiment of the invention DNA sequences to be recombined can consist of larger continuous stretches that contain more than a single coding sequence with intervening non-coding sequences, such as those that as may belong to a biosynthetic pathway or an operon. DNA sequences to be recombined can be sequences, which have already experienced one or more recombination events, for example homologous and/or non-homologous recombination events.

The recombination substrates can comprise non-mutated wild-type DNA sequences and/or mutated DNA sequences. In a preferred embodiment therefore it is possible to recombine wild-type sequences with already existing mutated sequences in order to evolve new mutated sequences.

In a preferred embodiment of the inventive process the bacteriophage or plasmid containing the promoter flanked by the two recombination substrates is generated by inserting fragments, each of which comprises one of the two recombination substrates, into the respective vector by genetic engineering methods. The fragments, each of which comprises one recombination substrate, can be obtained for example, by cutting a DNA molecule such as a plasmid comprising one of the two DNA sequences to be recombined with one or two appropriate restriction enzymes. Thereby a fragment is obtained comprising the respective DNA sequence to be recombined flanked by ends such as blunt ends or overhanging ends enabling the insertion of the fragment in the desired orientation into the bacteriophage or plasmid previously cut with one or two restriction enzymes and having identical ends. The fragments to be inserted also can be obtained by PCR amplification, whereby afterwards the PCR products can also be cut with restriction enzymes.

In another preferred embodiment of the inventive process the bacteriophage or plasmid containing the promoter flanked by the two recombination substrates is generated by homologous recombination of fragments comprising the respective recombination substrates. In this case the fragments to be recombined are flanked by sequences homologous to sequences of the bacteriophage or plasmid enabling the homologous recombination of the fragments into the vector leftward and rightward of the promoter.

After introduction of the bacteriophage or plasmid comprising the two recombination substrates into a host cell and incubation of the host cell containing the respective vector under selective conditions, that only allow the propagation of the cell and/or the bacteriophage if the promoter is oriented such the gene product of a marker gene is expressed, the progeny of the bacteriophage comprising the recombined DNA sequences is isolated. Depending on whether which selection strategies was chosen, i.e detection of recombinants during the lytic phase or as bacterial lysogens, the bacteriophage progeny, comprising recombined DNA sequences is isolated either from plaques or from bacterial lysogens.

After isolation of the bacteriophage progeny, the first and the second recombined DNA sequences contained in the bacteriophage progeny of the first bacterial host cell and/or the third and fourth recombined sequences contained in the bacteriophage progeny of the second bacterial host cell can be isolated and/or analysed. For example, the recombined DNA sequences can be isolated by PCR amplification and/or by restriction enzyme cleavage. If the recombined DNA sequences encode a protein, the isolated recombined DNA sequences can be sequenced and/or inserted in an expression vector under the functional control of one or more appropriate regulatory units in order to generate in an appropriate host cell the gene product. If the recombined DNA sequence comprise non-coding sequences with regulatory functions, the isolated recombined DNA sequences can be sequenced and/or inserted in an expression vector comprising a reporter gene, in order to study their regulatory effects on the expression of that reporter gene.

Therefore, the present invention also relates to a process for generating a hybrid or mosaic gene in a system comprising a bacteriophage and a bacterial host cell, wherein the inventive process for generating and detecting recombinant DNA sequences is carried out and the thus obtained hybrid or mosaic gene is selected and/or isolated from the bacteriophage progeny contained in the bacterial cell or in a plaque formed on a lawn of the bacterial cell. According to the inventive process the isolated hybrid gene is analysed and/or inserted into an expression vector under the functional control of at least one regulatory unit.

The present invention also relates to a hybrid gene which can be obtained by the inventive process for generating and detecting recombinant DNA sequences and/or the inventive process for generating a hybrid or mosaic gene.

The present invention also relates to a process for producing a hybrid protein encoded by a hybrid gene in a system comprising a bacteriophage and a bacterial host cell, wherein the inventive process for generating and detecting recombinant DNA sequences and/or the inventive process for generating a hybrid gene is carried out resulting in the formation of a hybrid gene and wherein the hybrid protein encoded by the hybrid gene is selected and/or isolated from the bacterial cell or from a plaque formed on a lawn of the bacterial cell upon expression. In one embodiment of the invention therefore, the hybrid protein encoded by the hybrid gene can be selected in the plaque and/or can be isolated therefrom, in case the lytic selection strategy was chosen. In case the selection strategy is based on bacterial lysogens, the hybrid protein can be selected in the bacterial lysogen and/or be isolated therefrom. In another embodiment of the inventive process the hybrid protein is selected and/or isolated by isolating the hybrid gene encoding the hybrid protein, inserting the gene into an expression vector under the functional control of at least one regulatory unit and introducing the expression vector into a suitable host cell. Then the host cell comprising the expression vector is cultivated under conditions which allow for the expression of the hybrid protein. Under appropriate conditions the hybrid protein can then be expressed, selected, isolated and/or analysed.

The present invention also relates to a protein, which is encoded by a hybrid gene and which is obtainable by the inventive process for producing a hybrid protein.

The present invention furthermore relates to bacteriophage lambda construct which comprises the promoter Pro, flanked by the Spec^{R} marker and the Cm^{R} marker, wherein are arranged at least a first and a second restriction site between the promoter and the Spec^{R} marker for inserting a first foreign DNA sequence and at least a third and a fourth restriction site between the promoter and the Cm^{R} for inserting a second foreign DNA sequence.

The present invention furthermore relates to plasmid pMIX-LAM, which is a derivative of plasmid pACYC184 that contains the pL+N promotor region and the flanking sequences cI+rexa and cIII+IS10 of bacteriophage lambda. pMIX-Lam contains furthermore a Cm^{R} gene. The vector also contains the multicloning sites MCS1 and MCS2, which flank the promoter containing pL+N fragment of lambda for inserting foreign DNA sequences. Plasmid DNA containing two DNA sequences to be recombined is cut with appropriate restriction enzymes in the lambda flanking regions cI and cIII to yield a fragment that contains the recombination substrates and that can be targeted to the lambda genome in a recipient host lysogen.

The present invention also relates to plasmid pAC-OX-OY, which is derived from a low copy number plasmid and which contains the colE1 replication origin. Plasmid pAC-OX-OY contains the two resistance markers Spec^{R} and Cm^{R} which flank the two recombination substrates and the targeting sequences LG and LD located at the ends of the recombination substrates. The targeting sequences promote integration into a lambda prophage genome. Linear DNA fragments containing the recombination substrates are obtained by enzymatic restriction and purification or by PCR amplification of the cassette.

The present invention also relates to a kit which can be used for carrying out the inventive processes. According to a preferred embodiment of the invention the kit comprises at least a first container which comprises DNA of bacteriophage lambda, wherein the phage comprises the promoter pL and the gam gene, or cells of an E. coli recA⁻ strain containing that bacteriophage, a second container which comprises cells of an E. coli recA⁻ strain and a third container comprising cells of an E. coli P2 lysogenic strain.

Another embodiment of the invention relates to a kit comprising at least a first container which contains DNA of plasmid pMIX-LAM or cells of an E. coli recA⁻ strain containing plasmid pMIX-LAM, a second container which comprises cells of an E. coli recA⁻ strain and a third container comprising cells of an E. coli P2 lysogenic strain.

Still another embodiment of the invention relates to a kit comprising at least a first container which contains DNA of a bacteriophage lambda, whereby the phage comprises the promoter Pro, flanked by the Spec^{R} marker and the Cm^{R} marker, or cells of an E. coli strain containing this bacteriophage and a second container which comprises cells of an E. coli strain.

Another embodiment of the invention relates to a kit comprising at least a first container which comprises DNA of plasmid pAC-OX-OY or cells of an E. coli strain containing plasmid pAC-OX-OY and a second container which comprises cells of an E. coli strain.

According to the invention the cells of the E. coli strains contained in the kits are mutS⁻.

The present invention also relates to the use of plasmid pMIX-LAM, plasmid pAC-OX-OY, a bacteriophage lambda comprising the promoter pL and the gam gene or a bacteriophage lambda comprising the promoter Pro, flanked by the Spec^{R} marker and the Cm^{R} marker, in the inventive process for generating and/or detecting recombinant DNA sequences, in the inventive process for generating a hybrid gene or in the inventive process for producing a hybrid protein.

The present invention is illustrated by the following figures and examples.

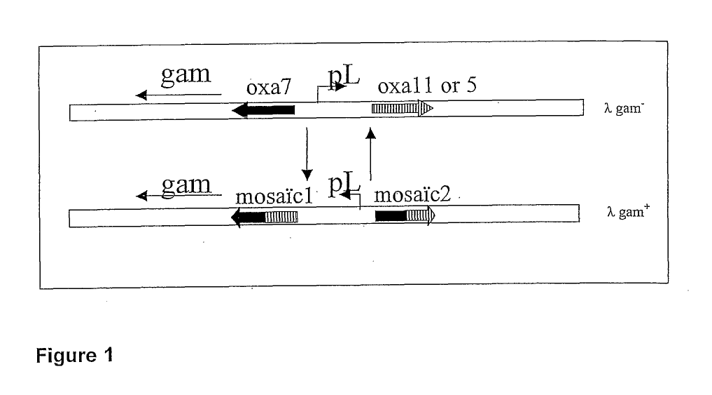

FIG. 1 shows the principle of the lytic selection strategy. Recombination substrates (the Oxa7-Oxa11 or Oxa7-Oxa5 gene pairs) are cloned in inverted orientation flanking the pL promoter. The lambda gam gene is located downstream of the introduced Oxa7 sequence. Phage in which pL is transcribed rightward are gam− and can be propagated lytically on P2 lysogens but not on E. coli recA− cells. Phage in which pL is transcribed leftward are gam+ and can be propagated lytically on E. coli recA− cells but not on P2 lysogens. Crossovers involving the inserted recombination substrates are accompanied by inversion of pL, and hence recombinants can be selected on appropriate hosts. The strategy is iterative, in that multiple rounds of recombination can be carried out.

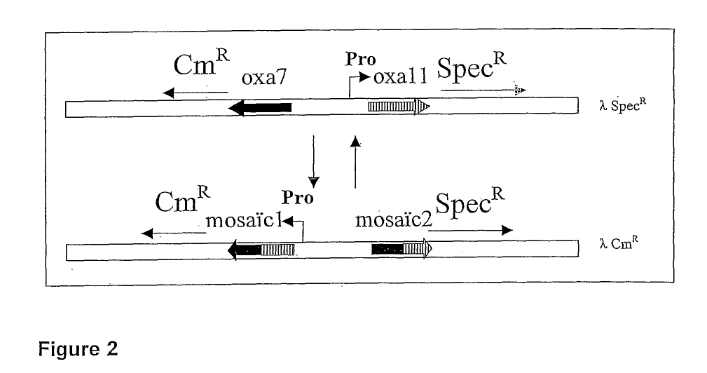

FIG. 2 shows the principle of the lysogenic selection strategy. Recombination substrates (shown is the Oxa7-Oxa11 gene pair) are cloned in inverted orientation flanking the Pro promoter. Genes conferring antibiotic resistance (here, chloramphenicol and spectinomycin) are located downstream of the Oxa sequences. Lysogens in which Pro is transcribed rightward can be selected on spectinomycin-containing media, and lysogens in which Pro is transcribed leftward can be selected on chloramphenicol-containing media. Crossovers involving the inserted recombination substrates are accompanied by inversion of Pro and can be selected in lysogens plated on appropriate antibiotics. The strategy is iterative, in that multiple rounds of recombination can be carried out.

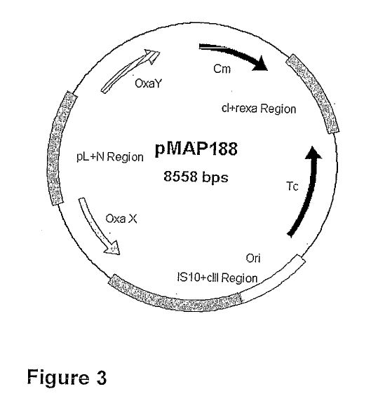

FIG. 3 shows the vector pMAP188, for use in the lytic selection strategy. Recombination substrates (OxaX and OxaY) are introduced into sites that flank the promoter-containing pL+N fragment of lambda. The resulting plasmid DNA is digested with enzymes that cut in the lambda flanking regions cI and cIII to yield a fragment that contains the shuffling cassettes and which can be targeted to the lambda genome in a recipient host lysogen.

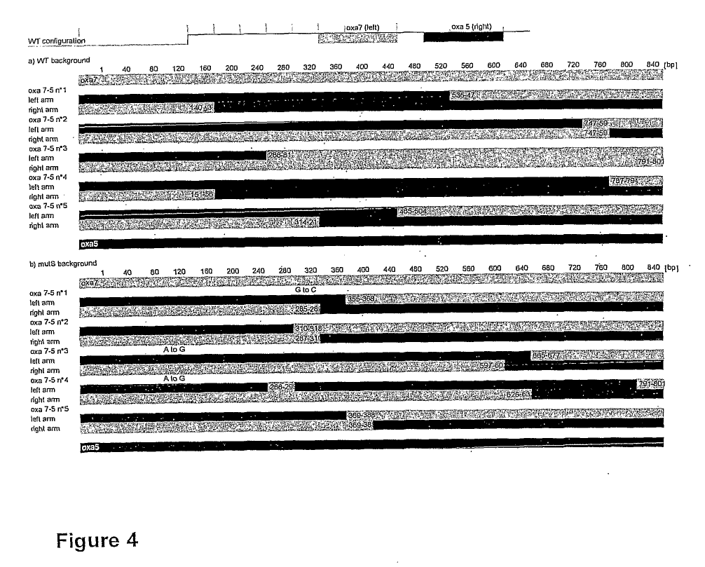

FIG. 4 shows a schematic alignment of pairs of λgt11 oxa7-5 “flip” recombinants obtained by the lytic selection strategy, a) Recombinants obtained in the wildtype background, b) Recombinants obtained in the mutS background. Oxa7 sequence, gray; Oxa5 sequence, black. The interval of identical sequence between Oxa7 and Oxa5 is indicated by the region of point mutation shown over the bars.

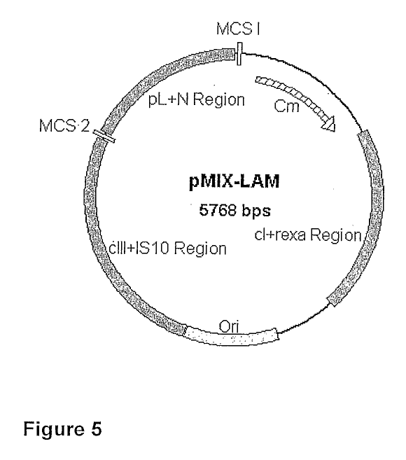

FIG. 5 shows the vector pMIX-LAM, for use in the lytic selection strategy. Genes to be shuffled are inserted into the multicloning sites MCS1 and MCS2, which flank the promoter-containing pL+N fragment of lambda. The resulting plasmid DNA is digested with enzymes that cut in the lambda flanking regions cI and cIII to yield a fragment that contains the shuffling cassettes and which can be targeted to the lambda genome in a recipient host lysogen.

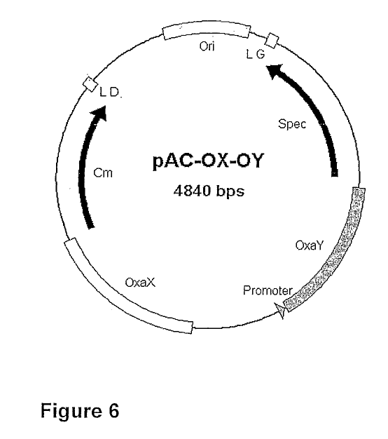

FIG. 6 shows a general schematic of vector pAC-OX-OY for use in the lysogen selection strategy, containing two recombination substrates (OxaX and OxaY). This plasmid is derived from a low copy number plasmid with a colE1 replication origin. Two resistance markers (here, Spectinomycin and Chloramphenicol) flank the genes to be shuffled. Targeting sequences (LG and LD) that promote specific integration into the lambda prophage genome are located at the ends of the shuffling cassettes. Linear DNA fragments containing the shuffling cassettes, are obtained by enzymatic restriction and purification or by PCR amplification of the cassette.

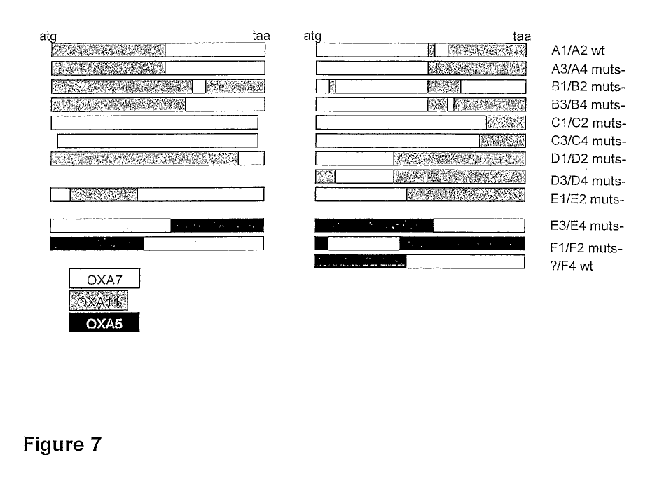

FIG. 7 shows the results of a sequence analysis of recombinant Oxa7-Oxa11 and Oxa7-Oxa5 gene pairs obtained by the lysogenic selection strategy. (In two cases sequence information is missing at the extreme ends of the ORF).

### EXAMPLES

### General Strategy

In order to create mosaic genes with a high efficiency in vivo, two selection strategies were developed. Both systems make use of constructs in which the two recombination substrates flank a promoter in an inverted configuration. Depending on the orientation of the promoter, one or the other of the two recombination substrates is transcribed, and genes further downstream of the substrates are similarly under this transcriptional control. The expression of these downstream genes can be detected and selected for under appropriate conditions, thereby allowing a specific promoter orientation to be selected. Since crossover recombination involving the two recombination substrates leads to promoter inversion, recombinants can be identified under conditions that select for the expression of specific downstream genes.

### a) Lytic Selection Strategy

The system based on the lytic selection strategy allows for the detection of recombinants during the lytic phase. Diverged sequences are cloned as shown in FIG. 1. Selection is based on expression or absence of expression of the lambda gam gene. In one orientation of the intervening sequence, transcription from the lambda promotor pL activates the gam gene, which allows plaque formation on an E. coli recA− lawn and prevents plaque formation on an E. coli P2 lysogen lawn. When pL is present in the opposite orientation, the absence of gam transcription allows lytic growth on the P2 lysogen and prevents growth on the recA− host.

### b) Lysogenic Selection Strategy

In this system, recombinants are recovered as bacterial lysogens—cells that harbor the lambda genome in their chromosome—rather than as plaques. Instead of activating transcription of the gam gene, in one orientation the artificial promoter Pro activates a gene expressing an antibiotic resistance marker (here, spectinomycin), and in the other orientation it activates another expressing an antibiotic resistance gene (here, chloramphenicol; see FIG. 2).

The two lambda-based strategies were tested for their ability to recombine pairs of divergent sequences in both wild type and MMR-defective E. coli strains. Three homeologous genes encoding the beta-lactamases Oxa7, Oxa11 and Oxa5 were chosen as recombination substrates to test the two systems. The Oxa11 and Oxa7 nucleotide sequences diverge by 4.5%, and the Oxa5 and Oxa7 sequences diverge by 22%. In both cases, recombination cassettes consisting of the two recombination substrates flanking an invertible promoter were constructed in plasmids and then transformed into an appropriate host lysogen to create starting lysogens containing these cassettes. These lysogens were subjected to conditions that initiate the lambda lytic cycle, resulting in the release of phage in which rolling circle-mediated recombination had occurred. Recombinant sequences were selected according to methods specific for each system and characterized by sequencing. The iterative nature of the system was demonstrated by using phage bearing recombination cassettes with mosaic sequences to initiate a new round of recombination.

The organism JM105 2Xlambda6T11 pMIX-LAM was deposited by MIXIS France S.A., Paris at the Deutsche Sammlung von Mikroorganismen und Zellkulturen GmbH, Braunschweig, Germany (DSMZ) on the 20 Jun. 2005: DSM 17434. The organism JM105 pAC-OX-OY (AA) was deposited by MIXIS France S.A., Paris at the DSMZ on the 20 Jun. 2005: DSM 17435.

### Methods and Materials

### Strains Used

The E. coli strains used are listed in Table 1.

TABLE 1
E. coli strains

| Strains | Genotype | Reference or source |
|---|---|---|
| AB1157 | thr1 leu6 proA2 his4, | ATCC |
| | thil argE3 lacY1 | |
| | galK2 ara14 xyl15 | |
| | mtl1 tsx33 str31 supE44thr⁺ | |
| AB1157 CI854 | AB1157 + prophage | MIXIS strain collection |
| | λCI854 | |
| C600 | thi-1 thr-1 leuB6 | DSMZ |
| | lacY1 tonA21 supE44 | |
| C600hfl⁻(*) | C600 + hflA150 | DSMZ |
| | (chr.:Tn10) | |
| C600 recA (*) | C600 + recA⁻ | M. Radman |
| NK5196 (P2) (*) | QI supII Tl⁻ lac⁻ | N. Kleckner |
| JM105 | endA1 thi rpdL | ATCC |
| | sbcB15 hsdR4 Δ(lac- | |
| | pro-AB) (F′ traD36 | |
| | proAB laclqZ ΔM15) | |
| JM105 (gt11X2) | JM105 + 2 prophages | M. Radman |
| | λGT11 | |

(*) mutS derivatives of these strains were generated by transduction

Introduction of Recombination Cassettes into Lambda Lysogens and Primary Phage Stock Production

For both selection strategies, plasmids containing recombination cassettes were digested with appropriate restriction enzymes to produce linear DNA fragments flanked by sequences homologous to a target lambda prophage. E. coli AB1157-λCI854::pKD46 cells were made competent and transformed with purified linear DNA. Prior to the induction of competence, cells were treated with L-arabinose, which promotes transcription of the red-gam complex encoded on pKD46. This complex mediates the integration of the shuffling cassettes into the prophage genome by homologous recombination (Kirill A. et al, PNAS 2000, 97, 6640-6645). Lysogens bearing integrated shuffling cassettes were selected in the presence of appropriate antibiotics at 30° C. For phage stock production, lysogens were cultured in liquid media at permissive temperature until OD=0.2. The cultures were shifted to 42° C. for 10 min and then to 37° C. until lysis was complete. After centrifugation, chloroform (1/500) was added to the supernatant, and the resulting phage stocks were stored at 4° C.

Selection of Recombinants with the Lytic Selection Strategy

Wild type and mutS P2 lysogens (NK5196 [P2] derivatives) were infected with primary phage stocks and plated on rich media to obtain plaques. To select first round recombinants (“flip”), phages were prepared from these plaques and used to infect C600 recA cells and NK5196 (P2) lysogens. To select second round recombinants (“flop”), phages were prepared from plaques that arose on the recA host and used to re-infect C600 recA cells and NK5196 (P2) lysogens. The relative frequency of plaques formed on each host was used to determine recombination frequencies.

Selection of Recombinants with the Lysogenic Selection Strategy

C600 hfl and C600 hfl mutS cells were infected with primary phage stocks and plated on spectinomycin to obtain resistant lysogens. For first round recombinant selection, lysogens were induced to undergo lysis, and phage stocks were prepared and used to infect C600 hfl cells. Lysogens were selected on chloramphenicol or spectinomycin.

### Molecular Analysis of Shuffled Sequences

For both selection strategies, first round and second round recombinant molecules were amplified by PCR using specific primer pairs and sequenced by standard methods.

### Results

### Recombination in Lambda Using the Lytic Selection Strategy

### Example I

Plasmids containing shuffling cassettes with the Oxa7-Oxa7, Oxa7-Oxa11 and Oxa7-Oxa5 recombination substrates were constructed. FIG. 3 shows the structure of plasmid pMAP188 containing two different Oxa substrates. The cassettes were excised from plasmids and introduced into host lysogens, which were then used to produce primary phage stocks. Lysogens containing two different lambda derivatives, λgt11 (Young, R A and Davis, R W, 1983 PNAS 80: 1194-1198) and λc/857 (Hendrix, R W et al. (eds) in Lambda II, 1983, CSH), were used as hosts for recombination studies. Table 2 shows that recombinants can be generated using both lambda derivatives and that depending on the extent of Oxa divergence and the lambda host, frequencies are generally ten-fold higher in the mutS background than in the wild type background.

TABLE 2
“Flip” recombination frequencies obtained with λgt11 and
λcI857 hosts in wild type and mutS backgrounds. Frequencies of
recombination were calculated as (viable count gam⁺)/(viable
count gam⁺ + viable count gam⁻) and expressed as the
mean ± standard deviation of three independent experiments.

| | Oxa gene | % | | |
|---|---|---|---|---|
| lambda | pair | divergence | freq WT | Freq mutS |
| λgt11 | 7-7 | 0 | 3.7 ± 1.1 × 10⁻⁴ | 1.4 ± 0.4 × 10⁻³ |
| | 7-11 | 4 | 8.6 ± 5.1 × 10⁻⁶ | 7.4 ± 0.9 × 10⁻⁴ |
| | 7-5 | 22 | 8.0 ± 4.8 × 10⁻⁶ | 4.1 ± 1.5 × 10⁻⁵ |
| λcI857 | 7-7 | 0 | 9.9 ± 1.1 × 10⁻³ | 1.4 ± 0.4 × 10⁻² |
| | 7-11 | 4 | 7.5 ± 2.0 × 10⁻⁴ | 5.5 ± 1.5 × 10⁻⁴ |
| | 7-11* | 4 | 1.4 ± 0.6 × 10⁻³ | 9.9 ± 9.0 × 10⁻³ |
| | 7-5 | 22 | 1.2 ± 0.3 × 10⁻⁶ | 8.7 ± 1.0 × 10⁻⁶ |

*Results of an experiment in which the recombination frequency of the Oxa7-Oxa11 gene pair was determined using a more sensitive protocol.

Forty-six recombinant Oxa pairs were isolated after both the “flip” and “flop” cycles of recombination and sequenced (22 Oxa7-Oxa11; 24 Oxa7-Oxa5). FIG. 4 shows in schematic form an example of recombined Oxa genes obtained from an Oxa7-Oxa5 substrate pair in the λgt11 host after a first round of recombination. The diversification of the recombination substrates was efficient. No obvious recombination hotspots were identified: identical recombination products were recovered in only three cases out of the 42 “flip” recombinants isolated (all Oxa7-Oxa5 recombinants). Very short intervals of sequence identity are sufficient to allow recombination (see e.g. FIG. 4b oxa 7-5 no. 1). In the mutS background recombination was also accompanied by the introduction of point mutations. As expected, a second cycle of recombination (“flop”) resulted in increased diversification of the substrate genes.

These results show that the lambda phage system can efficiently recombine diverged sequences. The overall recombination frequencies under the conditions used were surprisingly high. This is especially true for recombination in the context of the lambda red and gam genes, where frequencies reached 10⁻³ (see Table 3).

TABLE 3
“Flop” recombination frequencies obtained with λgt11 and
λcI857 in wildtype and mutS backgrounds. The Oxa7-11 and Oxa7-5
substrate pairs were constructed with two “flip” recombination products
having different lengths of segments of identical sequence.

| | Oxa gene | % | | | | | |
|---|---|---|---|---|---|---|---|
| λ | pair | divergence | | WT | | MutS | |
| λgt11 | 7-7 | 0 | sz | | | | |
| | | | freq | 1.0 × 10⁻³ | | 2.7 × 10⁻³ | |
| | 7-11 | 4 | sz | 332 bp | 100 bp | 332 bp | 100 bp |
| | | | freq | 5.6 × 10⁻³ | 2.9 × 10⁻³ | 7.7 × 10⁻³ | 6.3 × 10⁻³ |
| | 7-5 | 22 | sz | 83 bp | 1 bp | 83 bp | 1 bp |
| | | | freq | 4.0 × 10⁻³ | 2.5 × 10⁻³ | 3.0 × 10⁻³ | n.d. |

bp: base pairs;
sz: size of identical sequence;
n.d.: not determined.

Together with the diversification of the substrate genes, observed on the sequence level, these results indicate that the lambda tool can be exploited to create large libraries of diversified genes in directed evolution experiments.

### Recombination in Lambda Using the Lytic Selection Strategy

### Example II

Since the vector pMAP188 (see FIG. 3) is large, appears to be toxic to host bacteria, and does not have suitable restriction sites for further cloning, a new plasmid, pMIX-LAM (see FIG. 5), was constructed. Two critical features were incorporated into this construct: 1) the new vector contains several clusters of lambda sequences, including the invertible promoter and genes that encode essential lambda functions and also allow targeting of the shuffling cassette to a prophage genome; and 2) the vector provides unique sites for easy sub-cloning, and these sites can be exchanged for other multicloning sites to facilitate the introduction of more complex genes or gene clusters. pMIX-LAM is a pACYC184 derivative that includes the invertible lambda pL promoter region flanked by multicloning sites, obtained as an amplification product using pMAP188 as a template. It also includes the cI and cIII flanking sequences, isolated as restriction fragments from pMAP188.

### Recombination in Lambda Using the Lysogen Selection Strategy

In this approach, the identification of recombinants depends on the selection of individual cells (lysogens containing the shuffling cassettes) in which an artificial promoter situated between the two recombination substrates switches orientation, allowing one or the other of two antibiotic resistance markers downstream of the recombination substrates to be expressed. FIG. 6 describes the essential traits of vectors with a shuffling cassette containing genes to be recombined.

Shuffling cassettes containing the Oxa7-Oxa7, Oxa7-Oxa11 and Oxa7-Oxa5 recombination substrates were constructed. After integration of the shuffling cassettes into recipient lysogens, phage stocks were obtained by inducing lysis. Phage stocks were used to infect wild type and MMR-deficient E. coli shuffling strains. These strains also have the hflB mutation, which promotes a higher yield of lysogens (Herman, C. et al. 1993. PNAS. 90: 10861-10865). New lysogens were then recovered by selection on plates containing appropriate antibiotics. Recombined Oxa7-Oxa11 and Oxa7-Oxa5 gene pairs were recovered from lysogens selected on chloramphenicol and sequenced.

The sequences of chloramphenicol-resistant clones showed that all of them were recombinant, with different degrees of mosaicism (see FIG. 7). All of the sequenced ORFs are full-length and potentially code for functional proteins. Point mutations were observed in four recombinant sequences obtained from the MMR-deficient background (mutS−). It is noteworthy that recombinants involving the highly diverged genes (Oxa7-Oxa5, 22% divergence) were isolated.

1. Process for generating and detecting recombinant DNA sequences in a system comprising a bacteriophage and a bacterial host cell, wherein the bacteriophage contains a promoter flanked by a first and a second DNA sequences to be recombined and at least a first marker gene, located downstream of the first DNA sequence, wherein recombination between the two DNA sequence leads to an inversion of the promoter in a flip-flop manner and wherein depending on the orientation of the promoter one or the other of the DNA sequences and the marker gene can be transcribed or not, comprising the steps of:
a) incubation of a first bacterial host cell containing the bacteriophage under selective conditions, that only allow the propagation of the cell and/or of the bacteriophage if the promoter is oriented such that the gene product of the first marker gene is expressed, and
b) isolation of the bacteriophage progeny derived from the first host cells grown and/or propagated under selective conditions and containing a first and a second recombined DNA sequences,
wherein the first and second DNA sequences to be recombined diverge by more than 0.1%.

2. Process according to claim 1, comprising further the steps of:
a) introduction of the bacteriophage progeny obtained in 1b) into a second bacterial host cell,
b) incubation of the second host cell containing the bacteriophage progeny under selective conditions, that effect recombination and that only allow the propagation of the cell and/or of the bacteriophage if the promoter is oriented such that the gene product of the first marker gene is not expressed, and
c) isolation of the bacteriophage progeny derived from the second host cells grown and/or propagated under selective conditions and containing a third and a fourth recombined DNA sequences.

3. Process according to claim 2, wherein further recombined DNA sequences are generated by subjecting the bacteriophage progeny obtained in 2c) at least once to another cycle of steps 1a) to 1b) or steps 1a) to 1b) plus steps 2a) to 2c).

4. Process according to claim 1, wherein the first host cells containing the bacteriophage are generated by introduction of the bacteriophage into a bacterial cell with or without a prophage in its genome.

5. Process according to claim 1, wherein the bacteriophage is a derivative of bacteriophage lambda.

6. Process according to claim 1, wherein the first host cell containing the bacteriophage is generated by introduction of a plasmid containing bacteriophage sequences, the two DNA sequences to be recombined flanking the promoter and the first marker gene into a bacterial cell containing a prophage in its genome.

7. Process according to claim 6, wherein upon introduction of the plasmid into the bacterial cell containing the prophage the plasmid integrates into the genome via homologous recombination.

8. Process according to claim 6, wherein the plasmid is plasmid pMIX-LAM, which is a derivative of plasmid pACYC184 including the pL+N promoter region and the flanking sequences cI+rexa and cIII+IS10 of bacteriophage lambda and which can be targeted to the lambda genome in a host lysogen.

9. Process according to claim 6, wherein the plasmid is pAC-OX-OY, which is derived from a low copy number plasmid and which contains the colE1 replication origin and the targeting sequences LG and LD that promote integration into a lambda prophage genome.

10. Process according to claim 1, wherein the promoter is the pL promoter of lambda.

11. Process according to claim 1, wherein the promoter is the promoter Pro.

12. Process according to claim 1, wherein the first marker gene is selected from the group consisting of a lambda gene, a nutritional marker gene, an antibiotic resistance marker gene and a sequence encoding a subunit of an enzyme.

13. Process according to claim 12, wherein the first marker gene is the gam gene of lambda.

14. Process according to claim 13, wherein the transcription of the gam gene from the promoter in flip position allows the formation of plaques on a lawn of Escherichia coli recA host cells and prevents plaque formation on a lawn of E. coli P2 lysogenic host cells.

15. Process according to claim 13, wherein the absence of transcription of the gam gene due to the flop orientation of the promoter allows the plague formation on a lawn of E. coli P2 lysogenic host cells end prevents the plague formation on a lawn of E. coli recA host cells.

16. Process according to claim 12, wherein the first marker gene is Cm^{R}.

17. Process according to claim 16, wherein the transcription of the Cm^{R} gene from the promoter in flip position allows the growth of the bacterial host cells on a medium containing chloramphenicol and the absence of transcription of the Cm^{R} gene due to the flop orientation of the promoter prevents the growth of the bacterial host cells on a medium containing chloramphenicol.

18. Process according to claim 1, wherein the bacteriophage comprises a second marker gene that is located downstream of the second DNA sequence to be recombined and that can be transcribed or not depending on the orientation of the promoter.

19. Process according to claim 2 wherein the bacteriophage further comprises a second marker gene that is located downstream of the second DNA sequence to be recombined and that can be transcribed or not depending on the orientation of the promoter, and wherein the second host cells containing the bacteriophage progeny with the second marker gene are incubated under selective conditions that only allow the propagation of the cell and/or of the bacteriophage if the promoter is orientated such that the gene product of the second marker gene is expressed.

20. Process according to claim 18, wherein the second marker gene is selected from the group consisting of a nutritional marker gene, an antibiotic resistance marker gene and a sequence encoding a subunit of an enzyme.

21. Process according to claim 20, wherein the second marker gene is Spec^{R}.

22. Process according to claim 21, wherein the transcription of the Spec^{R} gene from the promoter in flop position allows the growth of the bacterial host cells on a medium containing spectinomycin and the absence of the transcription of the Spec^{R} gene due to the flip orientation of the promoter prevents the growth of the bacterial host cells on a medium containing spectinomycin.

23. Process according to claim 1, wherein the bacterial host cell is a cell of a gram-negative bacterium, a gram-positive bacterium or a cyanobacterium.

24. Process according to claim 23, wherein the gram-negative bacterium is E. coli.

25. Process according to claim 1, wherein the bacterial host cell has a functional mismatch repair system.

26. Process according to claim 1, wherein the bacterial host cell is transiently or permanently deficient in the mismatch repair system.

27. Process according to claim 26, wherein the transient or permanent deficiency of the mismatch repair system is due to a mutation, a deletion, and/or an inducible expression or repression of one or more genes involved in the mismatch repair system, a treatment with an agent that saturates the mismatch repair system and/or a treatment with an agent that globally knocks out the mismatch repair.

28. Process according to claim 26, wherein the bacterial cells has a mutated mutS gene and/or a mutated mutL gene.

29. Process according to claim 1, wherein the first and the second DNA sequences to be recombined diverge by at least two nucleotides.

30. Process according to claim 1, wherein the first and the second DNA sequences to be recombined are naturally occurring sequences and/or artificial sequences.

31. Process according to claim 30, wherein the first and/or the second DNA sequences to be recombined are derived from viruses, bacteria, plants, animals, and/or human beings.

32. Process according to claim 1, wherein each of the first and the second DNA sequences to be recombined comprises one or more protein-coding sequences and/or one or more non-coding sequences.

33. Process according to claim 1, wherein the insertion of the first and/or the second DNA sequence to be recombined into the bacteriophage carried out by cloning a fragment comprising the respective DNA sequences into a site of the bacteriophage previously cut with at least one restriction enzyme.

34. Process according to claim 1, wherein the insertion of the first and/or the second DNA sequence to be recombined into the bacteriophage is carried out by homologous recombination of a fragment comprising the respective DNA sequence and flanked by sequences homologous to sequences of the bacteriophage.

35. Process according to claim 1, wherein the bacteriophage progeny comprising recombined DNA sequences is isolated from plaques.

36. Process according to claim 1, wherein the bacteriophage progeny comprising recombined DNA sequences is isolated from bacterial lysogens.

37. Process according to claim 2, wherein the first and the second recombined DNA sequences contained in the bacteriophage progeny of the first bacterial host cell and/or the third and fourth recombined sequences contained in the bacteriophage progeny of the second bacterial host cell are isolated and/or analysed.

38. Process according to claim 37, wherein the recombined DNA sequences are amplified by PCR and/or isolated by restriction enzyme cleavage.

39. Process for generating a hybrid gene in a system comprising a bacteriophage and a bacterial host cell, wherein the process according to claim 1 is carried out and the thus obtained hybrid gene is selected and/or isolated from the bacteriophage progeny contained in the bacterial cell or in a plague formed on a lawn of the bacterial cell.

40. Process according to claim 39, wherein the isolated hybrid gene is analysed and/or inserted into an expression vector under the functional control of at least one regulatory unit.

41. Process for producing a hybrid protein encoded by a hybrid gene in a system comprising a bacteriophage and a bacterial host cell, wherein the process according to claim 1 is carried out resulting in the formation of a hybrid gene and wherein the hybrid protein encoded by the hybrid gene is selected and/or isolated from the bacterial cell or from a plaque formed on a lawn of the bacterial cell upon expression.

42. Process according to claim 41, wherein the hybrid gene encoding the hybrid protein is isolated and inserted into an expression vector under the functional control of at least one regulatory unit.

43. Process according to claim 42, wherein the expression vector comprising the inserted hybrid gene is introduced into an appropriate host cell.

44. Process according to claim 43, wherein the host cell comprising the expression vector is cultivated under conditions which allow for the expression of the hybrid protein.

45. Hybrid gene obtainable by a process according to claim 39.

46. Protein, which is encoded by a hybrid gene according to claim 45 and which is obtainable by a process according to claim 41.

47. Derivative of bacteriophage lambda which composes the promoter Pro, flanked by the Spec^{R} marker and the Cm^{R} marker, wherein at least a first and a second restriction site are arranged between the promoter and the Spec^{R} marker for inserting a first foreign DNA sequence, and at least a third and a fourth restriction site are arranged between the promoter and the Cm^{R} marker for inserting a second foreign DNA sequence.

48. Plasmid, which is a derivative of plasmid pACYC184, which confess the pL+N promoter region and the flanking sequences cI+rexa and cIII+IS10 of bacteriophage lambda, the multifunciton sites MCS1 and MCS2 flanking the promoter containing pL+N fragment and the Cm^{R} marker gene and which can be targeted to the lambda genome in a host system.

49. Plasmid, which is derived from a low copy number plasmid, which contains the colE1 replication origin, the marker genes Cm^{R} and Spec^{R} and the targeting sequences LG and LD, which promote intergration into a lambda prophage genome.

50. Kit, comprising at least a first container which comprises DNA of bacteriophage lambda comprising the promoter pL and the gam gene, or cells of an E. coli recA strain containing that bacteriophage, a second container which comprises cells of an E. coli recA strain and a third container comprising cells of an E. coli P2 lysogenic strain.

51. Kit, comprising at least a first container which comprises DNA of plasmid according to claim 48 or cells of an E. coli recA strain containing plasmid according to claim 48, a second container which comprises cells of an E. coli recA strain and a third container comprising cells of an E. coli P2 lysogenic strain.

52. Kit, comprising at least a first container which comprises DNA of a bacteriophage derivative according to claim 47 or cells of an E. coli strain containing the bacteriophage derivative according to claim 47 and a second container which comprises cells of an E. coli strain.

53. Kit, comprising at least a first container which composes DNA of plasmid according to claim 49 or cells of an E. coli strain containing plasmid according to claim 49 and a second container which comprises cells of an E. coli strain.

54. Kit, according to claim 50, wherein the cells of the E. coli strains are mutS.

55. (canceled)

56. Kit according to claim 51, wherein the cells of the E. coli strains are mutS.

57. Kit according to claim 52, wherein the cells of the E. coli strains are mutS.

58. Kit according to claim 53, wherein the cells of the E. coli strains are mutS.
